(19) United States
(12) Patent Application Publication
(10) Pub. No.: US 2010/0286603 A1
(43) Pub. Date: Nov. 11, 2010

(54) KIT AND METHOD FOR PREPARATION OF A DEGARELIX SOLUTION

(76) Inventor: Carin WINDERSTRÖM, Hollviken (SE)

Correspondence Address: Finnegan/Ferring, B.V., 901 New York Avenue, N.W., Washington, DC, 20001, US

(21) Appl. No.: 12/774,113

(22) Filed: May 5, 2010

Related U.S. Application Data
(60) Provisional application No. 61/187,828, filed on Jun. 17, 2009.

(30) Foreign Application Priority Data
May 5, 2009 (EP) 09006116.9

(51) Int. Cl. A61M 5/32; A61J 1/14; A61M 5/30

(52) U.S. Cl. 604/68; 604/92; 604/416

(57) ABSTRACT

A kit and method for preparing a Degarelix solution for administration to a patient. The kit includes a first chamber containing lyophilised Degarelix, a second chamber containing water, a syringe for transferring the water from the first chamber to the second chamber, at least one of a hypodermic needle, a safety needle device, or a needleless device for delivery of the Degarelix to the patient, and a mixing apparatus. The mixing apparatus is configured to receive the first chamber to mix the Degarelix and water to produce a Degarelix solution suitable for deliver to a patient. The method includes transferring a volume of water from a first chamber into a second chamber containing lyophilised Degarelix, connecting the second chamber to a mixing apparatus and mixing the water and lyophilised Degarelix for a period of time.

This application claims priority to U.S. Provisional Application No. 61/187,828 filed Jun. 17, 2009, and European Patent Application No. 09006116.9 filed May 6, 2009, all of which are incorporated herein by reference in their entireties.

### FIELD OF INVENTION

The present disclosure relates to a kit and a method for the preparation of a Degarelix solution for administration to a patient.

### BACKGROUND

Degarelix is a gonadotropin-releasing hormone antagonist. When used in a clinical preparation, Degarelix has been shown to produce a reduction in levels of testosterone and is used as a treatment for prostate cancer.

In order to prepare a Degarelix solution for administration to a patient, the lyophilised drug product needs to be reconstituted with water for injection. Degarelix may not readily dissolve and a lengthy reconstitution process may be undertaken each time a care-provider needs to administer a dose of Degarelix to a patient.

At present, the drug product and water for injection are supplied in separate vials. Each vial is typically sealed with a pierceable lid that enables passage of a hypodermic needle into the vial.

FIG. 1 illustrates the current method of preparing a Degarelix solution and shows preparation steps labelled alphabetically, A to N.

Typically water for injection (WFI) 15 is provided in a sealed vial 10 comprising a cap 25 and a rubber stopper 20. As a first step (A) the cap 25 is removed from the vial and a reconstitution needle 30 is then coupled to a syringe 40 (B). The reconstitution needle is typically 21 G—with dimensions of 0.8 mm by 50 mm.

The reconstitution needle is then passed through the rubber stopper into the WFI vial (C) and a predetermined volume of WFI drawn into the barrel 46 of the syringe (D). The syringe and needle are then removed from the WFI vial (E).

Lyophilised Degarelix 55 is provided in a sealed vial 50 having a cap 65 and rubber stopper 60. The cap 65 is removed (G) and the reconstitution needle 30 is passed through the rubber stopper 60 (H).

The WFI in the syringe is then transferred into the Degarelix vial (I) and the assembly of vial, needle, and syringe is swirled by hand to ensure that the lyophilised Degarelix has completely gone into solution. The Degarelix drug product does not lend itself to reconstitution into an administrable solution and the time needed for reconstitution may vary considerably. In laboratory testing, the reconstitution times for hand swirled Degarelix solutions comprising 80 mg and 120 mg of lyophilised Degarelix are about 5 minutes and 7 minutes respectively. The slowest reconstitution times are considerably greater, however, and can be up to 15 minutes.

To ensure that the Degarelix drug product has been fully reconstituted, the care-provider is required to swirl the assembly, by hand, for a period of up to 15 minutes. To assess whether the drug has been reconstituted, the care-provider periodically checks the vial to determine whether the solution is completely clear or not.

After full reconstitution, a measured dose of the Degarelix solution is drawn into the chamber of the syringe (K) and the reconstitution needle and syringe are removed from the vial (L). The reconstitution needle is not suitable for performing a subcutaneous injection. Therefore, the needle 30 needs to be removed from the syringe (M) and replaced by a suitable (e.g., 25G or 27G) needle for subcutaneous injection 70 (N).

The total time taken to prepare each dose includes the time taken to transfer WFI from the water vial to the drug vial (which should not be rushed so as to avoid injury), to reconstitute the drug product, to withdraw the correct dosage and to change the needle to an appropriate length and gauge. It can be seen that it is not an efficient use of a care-provider's time to be preparing a Degarelix solution for what can, in total, be longer than 15 minutes prior to every administration to a patient. Furthermore, if something goes wrong in the process of delivering the solution to the patient, or if too much time passes between preparing a solution and accessing the patient, then a new solution needs to be prepared.

As different care-providers may swirl the assembly for different amounts of time, or with differing vigour, it is conceivable that differing degrees of dissolution of Degarelix occur prior to injection. If a patient receives less of a dose than prescribed then the treatment may not be as effective as it could be. The completeness of the reconstitution process is something that is normally assessed by the care-provider checking the visual clarity of the solution, so the patient will usually be given the correct dose even if it takes a considerable period of time for reconstitution.

In addition to the issue of consistency of product and the considerable amount of care-giver's time being spent in preparing the solution, there are safety issues involved with the preparation process. The process involves the use of a hypodermic needle to transfer water between vials and a different needle to administer the drug to a patient. This may increase the possibility for accidental needle stick incidents during preparation.

It is an aspect of this disclosure to provide kits and methods that improve the preparation of a Degarelix solution for administration to a patient.

### SUMMARY OF THE DISCLOSURE

The disclosure includes kits and methods for the preparation of a Degarelix solution. In a first aspect, the disclosure may include a kit for the preparation of a Degarelix solution for administration to a patient. The kit may include a first chamber containing a predetermined mass of lyophilised Degarelix, and a second chamber containing a predetermined volume of solvent such as water for injection (WFI). The kit may also include a device for transferring at least a portion of the WFI from the second chamber into the first chamber and an automatic mixing apparatus comprising an adaptor for receiving the first chamber. This may allow the contents of the first chamber, after WFI has been transferred into the first chamber, to be automatically mixed to form the Degarelix solution. The kit may further include a hypodermic needle for subcutaneous delivery of the Degarelix solution to the patient.

In an exemplary embodiment, the mass of lyophilised Degarelix may be between 10 mg and 300 mg, particularly between 15 mg and 240 mg. Likewise, the second chamber may contain between 1 ml and 10 ml of solvent (e.g., WFI), particularly between 2 ml and 6 ml of WFI. A convenient size of WFI vial contains 6 ml of water and, if a 6 ml vial is used, the appropriate volume of water may be withdrawn to give a desired concentration of drug after reconstitution.

The mass of lyophilised Degarelix and the volume of WFI can be any amount and volume. As an example, Degarelix may be administered with concentrations of 40 mg/ml for starter doses and 20 mg/ml for maintenance doses. Thus, a solution containing 20 mg/ml of Degarelix may be reconstituted using about 80 mg of Degarelix and about 4 ml of WFI. Likewise, a Degarelix solution of 40 mg/ml may be reconstituted using about 120 mg of Degarelix and about 3 ml of WFI. Specific kits may be assembled to prepare Degarelix solutions having those concentrations. It may be desirable to prepare Degarelix solutions with higher concentrations for particular applications. Thus, kits and methods according to the present disclosure may be used for the preparation of Degarelix solutions having a concentration of, for example, 60 mg/ml.

Different amounts of water and drug product may be used to give the same dosage. For example, a 20 mg/ml solution may be made from 80 mg of Degarelix and 4 ml of WFI or may be made from 88 mg of Degarelix and 4.2 ml of WFI. In the latter case, the slight increase in volume of solution reconstituted may enable a care provider to deliver a more exact 4 ml dose of the solution to a patient.

As a further example, a 60 mg/ml solution may be made from 240 mg of Degarelix with 4 ml of WFI or 180 mg of Degarelix with 3 ml of WFI.

Different doses may be formed from different ratios of the amount of Degarelix to the volume of WFI, and such different doses may be required for different treatment regimes or for treatment of different clinical conditions.

The kit may include more than one chamber containing lyophilised Degarelix and more than one chamber containing solvent, e.g., WFI. Thus, as an example, the kit may include one or more chambers containing a first mass of lyophilised Degarelix and one or more chambers containing a second mass of lyophilised Degarelix. Likewise, the kit may include one or more chambers containing a first volume of WFI and one or more chambers containing a second volume of WFI. The advantage of such a kit is that different doses of Degarelix may be easily prepared for administration to different patients, or for administration to the same patient at different stages in treatment.

Some treatment regimes may require more than one reconstitution of Degarelix to deliver a single dose to a patient. As an example, if a dose of Degarelix is 360 mg, this dose may be delivered in two injections each containing 180 mg of Degarelix at a concentration of 60 mg/ml. Thus, the kit may comprise more than one chamber containing lyophilised Degarelix so that more than one administration of Degarelix can be prepared at the same time, or in quick succession.

The automatic mixing apparatus may be a device such as a vortex mixer or swirler. Vortex mixers are generally used to produce a vortex in liquids held in test tubes and produce a mixing effect. The automatic mixing apparatus may include any suitable device for providing swirling or agitation of the water and Degarelix mixing to aid formation of a solution. The mixer may include a dial or other control for varying the intensity of the mixing. The automatic mixing apparatus may also include a preset or marked intensity to enable the care-provider to more consistently use a predetermined or desired intensity for reconstitution. It is contemplated that the intensity may be set such that a continuous agitation is established in the liquid or such that a vortex is formed within the first chamber, if a vortex mixer is used. It is also contemplated that a preset or marked intensity may also enable more consistent mixing by different care-providers. It is further contemplated that the mixing apparatus may also include a timer. The timer may include an alarm such as a buzzer or a light to indicate when a predetermined time lapses.

When mixing by hand, one person may be more diligent in mixing the solution for the prescribed length of time than another person, and different people may swirl or agitate the solution with different degrees of vigour. This may result in a wide range of times over which the Degarelix solution may be formed and may result in instances where the Degarelix is not fully reconstituted into solution until a considerable time has passed.

An automatic mixing apparatus may significantly decrease the time taken for preparation of the Degarelix solution. For example, laboratory tests have shown that average reconstitution times when an automatic swirling machine is used are in the region of 0.8 minutes and the slowest times for reconstitution are in the region of 1.25 to 1.5 minutes (tested for the 80 mg and 120 mg doses). This compares well with the present system of swirling by hand, where the slowest times for reconstitution are near to 15 minutes. An automatic mixing apparatus may enable a reconstitution protocol to be formulated in which the Degarelix and WFI are mixed for a relatively short period of time, after which the Degarelix should in the vast majority of cases have gone into solution, as compared with mixing by hand. It is contemplated that the care-provider may check the clarity of the solution after it has been reconstituted with the automatic mixing apparatus and, if fully reconstituted, administer the solution to the patient.

The mixer may include an adaptor that allows the first chamber to be coupled to the mixer so that its contents can be mixed. For example, the adapter may be in the form of a sleeve or guide that can attach to the mixer and receive the chamber such that mixing of liquid within the chamber can be effected.

The hypodermic needle for subcutaneous delivery to the patient may, for example, include a needle with a gauge of between 238 and 338, and in particular, a gauge of between 258 to 278. In such an arrangement, where the use of a hypodermic needle is avoided until the point at which the Degarelix solution is ready for administration to the patient, the opportunity of accidental needle stick is significantly reduced compared to the prior art method of preparing the solution.

It is contemplated that the hypodermic needle may include a safety needle device that may include, for example a needle shielded from the user and only extend beyond this shielding on a deliberate injection into a patient. The risks of accidental needle stick incidents are further reduced by the incorporation of such delivery means in the kit.

It is also contemplated that the hypodermic needle may instead include a completely needle-free device such as is known in the art.

In a first embodiment of the kit, the first chamber may be defined by a first vial and the second chamber may be defined by a second vial. Thus, the first chamber may be the space or volume within the wall of the first vial and the second chamber may be the space or volume within the wall of the second vial. The kit may additionally include a syringe and first and second vial adaptors for enabling needleless communication between each vial respectively and the syringe. The use of vial adaptors may reduce the risk of inadvertent needle sticks during the process of transferring WFI from the second vial to the first vial. It is contemplated that the vial adaptors may include internal spikes configured to pierce a septum or membrane sealing a respective vial. As is known in the art, the syringe may be coupled to the adaptors directly without use of an external needle.

In addition to being capable of transferring the water between vials, the syringe may also be used for withdrawing the solution formed in the first vial after mixing and delivering this solution to the patient. The syringe may therefore be connectable to, e.g., a hypodermic needle for subcutaneous delivery of the Degarelix solution to the patient.

It is contemplated that the vial adaptors may have one or more narrow channels for passage of WFI from the syringe, which may initiate turbulence when the WFI is added to the Degarelix and may initiate dissolution of the Degarelix. For example, such narrow passages may inject the WFI as a pressurized stream thereby increasing the velocity of WFI as it is injected into the Degarelix vial, which may add to initial turbulence in the vial and may accelerated dissolution of Degarelix.

The adaptor includes a sleeve mountable to the mixing apparatus and configured to receive the first vial such that the contents of the first vial are agitated by the mixing apparatus. For example, the sleeve may extend to at least half of the height of the vial. The adaptor may be coupled to the mixer in any suitable way. For example, the adaptor may have lugs that engage with recesses on the mixer, or vice versa. Alternatively, the adaptor may be clamped to the mixer or connected via any other method capable of transferring the mixing action of the mixer to the vial received in the adaptor.

In one embodiment, the adaptor may include a substantially tubular structure having a first end with a diameter capable of engaging with a tubular element on a mixing machine and a second end with a diameter capable of receiving the vial. If the automatic mixing apparatus is a vortex mixer or vortex swirler, the adaptor may include a substantially cylindrical guide that receives the first vial on the mixing plate of the mixer. On actuation of the vortex mixer, the guide spins and a vortex is instigated in the liquid within the first vial.

It is contemplated that the syringe may remain coupled to the first vial after transferring water from the second vial to the first vial and prior to withdrawing the solution formed in the first vial. In such a case, the syringe may remain attached to the vial during the mixing process. The kit may further comprise a guide sleeve for supporting the syringe in connection to the first vial while the first vial is received on the mixing apparatus. The guide sleeve may attach at a lower end to the outer surface of the first vial and support the syringe at an upper end.

In a second embodiment of the kit, the first chamber may be defined by a vial and the second chamber containing water for injection may be defined within the barrel of a syringe. Thus, the second chamber may be a syringe that is pre-filled or pre-loaded with WFI. In this kit arrangement, the prefilled syringe may be coupled to the first chamber by means of a vial adaptor as described above. For example, the step of withdrawing water from a separate water vial can be eliminated. Furthermore, the pre-filled syringe may be filled with the appropriate volume of water for reconstituting the Degarelix within the first container that reduces the possibility for error by the care provider preparing the solution.

Where the second chamber is a syringe pre-filled with water for injection, the same syringe can be used for withdrawing the Degarelix solution from the first chamber after reconstitution and administering the solution to a patient. Thus, the second chamber in these circumstances is couplable to a device for subcutaneous delivery of the Degarelix solution. Alternatively, the kit may be provided with a separate syringe for withdrawing reconstituted Degarelix solution from the first chamber and administering it to a patient.

In a third embodiment of the kit, the first and second chambers may be defined within a single multi-chambered syringe. Such multi-chambered syringes, for example dual-chambered syringes, are known in the art. In this arrangement, the lyophilised Degarelix drug product may be contained in a first chamber of the syringe, and the WFI in a second chamber of the syringe. The water may then be introduced to the lyophilised Degarelix by applying pressure to the plunger of the syringe such that the water is transferred from the second chamber to the first chamber via an inbuilt channel or port between the second and first chambers.

Where the first and second chambers are defined within a multi-chambered syringe, an adaptor may enable the multi-chambered syringe to be received by a mixing apparatus, such that the contents of the first chamber after liquid has been introduced into the first chamber, can be swirled or agitated by the mixing apparatus.

It is contemplated that the Degarelix solution may be prepared within the syringe, a hypodermic needle, safety needle or a needleless delivery device can be connected to the syringe for delivery to the patient.

Another aspect of the disclosure may include a method of preparing a Degarelix solution for administration to a patient comprising the steps of transferring a predetermined volume of WFI into a chamber containing a predetermined mass of lyophilised Degarelix and coupling the chamber to an automatic mixing apparatus to mix the contents of the chamber for a predetermined time. The method may further comprise the steps of removing the chamber from the mixing apparatus after the period of time, such that the chamber contains a Degarelix solution suitable for delivery to the patient. The Degarelix solution may then be delivered to the patient.

The chamber may be a vial containing the predetermined volume of Degarelix and transferring the volume of water may include the use of vials fitted with vial adapters allowing a syringe to be coupled to the vials. For example, the method may include coupling a syringe containing WFI to a vial containing lyophilised Degarelix via a needleless vial adapter and then injecting the WFI into the vial as described above with respect to the first embodiment of the kit. Alternatively, the chamber may be a first chamber in a multi-chambered syringe as described above with respect to the second embodiment of the kit. For example, the first chamber containing the predetermined volume of lyophilised Degarelix may be a chamber in a dual or double chambered syringe e.g., a multi-chambered syringe and the WFI may then be contained within a second chamber in the multi-chambered syringe.

The method may further include transferring the WFI through an entrance of the chamber under pressure, that is, as a pressurize stream. Such delivery under pressure may create a turbulent initial mixing with the lyophilised Degarelix, which may initiate the reconstitution reaction of Degarelix with the water such that the time for forming a Degarelix solution is reduced. For example, the WFI may be delivered through a narrow port or channel to increase the velocity of the water as it reaches the powdered drug product. As described above the WFI may be forced through one or more narrow slots defined in a vial adaptor or through a narrow port between chambers in a dual-chambered syringe.

The method may further include mixing the contents of the chamber for less than 5 minutes, particularly between 0.5 and 3 minutes, and more particularly between 0.8 and 1.5 minutes, for example about 1.25 minutes. It is contemplated that these mixing times may be long enough for full reconstitution of Degarelix in the majority of cases, however, the exact mixing time may vary depending on the concentration of Degarelix solution being formed.

The method may further include setting the intensity of mixing, which may for example be set by the user. It is contemplated that the mixing apparatus may have a single mixing intensity marked to reduce error or confusion by the user. Alternatively, the mixing apparatus may be adapted such that it only operates at a single intensity.

It is noted that the methods described above may be combined with other features described above in relation to other aspects of the invention, for example an automatic mixing apparatus. Likewise, the methods described above may be performed using one or more kit embodiments described above, for example a kit comprising vials and vial adaptors or a kit comprising a multi-chambered syringe.

### BRIEF DESCRIPTION OF THE DRAWINGS

Exemplary embodiments of the disclosure will now be described with reference to the Figures in which:

FIG. 1 is a diagram illustrating steps involved in a conventional method of preparing a Degarelix solution.

FIG. 2 illustrates some elements of a kit according to an embodiment of the disclosure.

FIG. 3 illustrates a safety needle for use with a kit according to disclosure of the invention.

FIG. 4 illustrates elements of a kit according to an embodiment of the invention while assembled for use.

FIG. 5 illustrates sectional and perspective views of an adaptor for use in a kit according to an embodiment of the disclosure.

FIG. 6 illustrates a sectional view of a vial mounted to a vortex mixer using an adaptor according to an embodiment of the disclosure.

FIGS. 7 to 11 illustrate method steps involved in using the kit according to an embodiment of the disclosure.

FIG. 12 illustrates a perspective view of a dual-chambered syringe for use in a kit according to an embodiment of the disclosure.

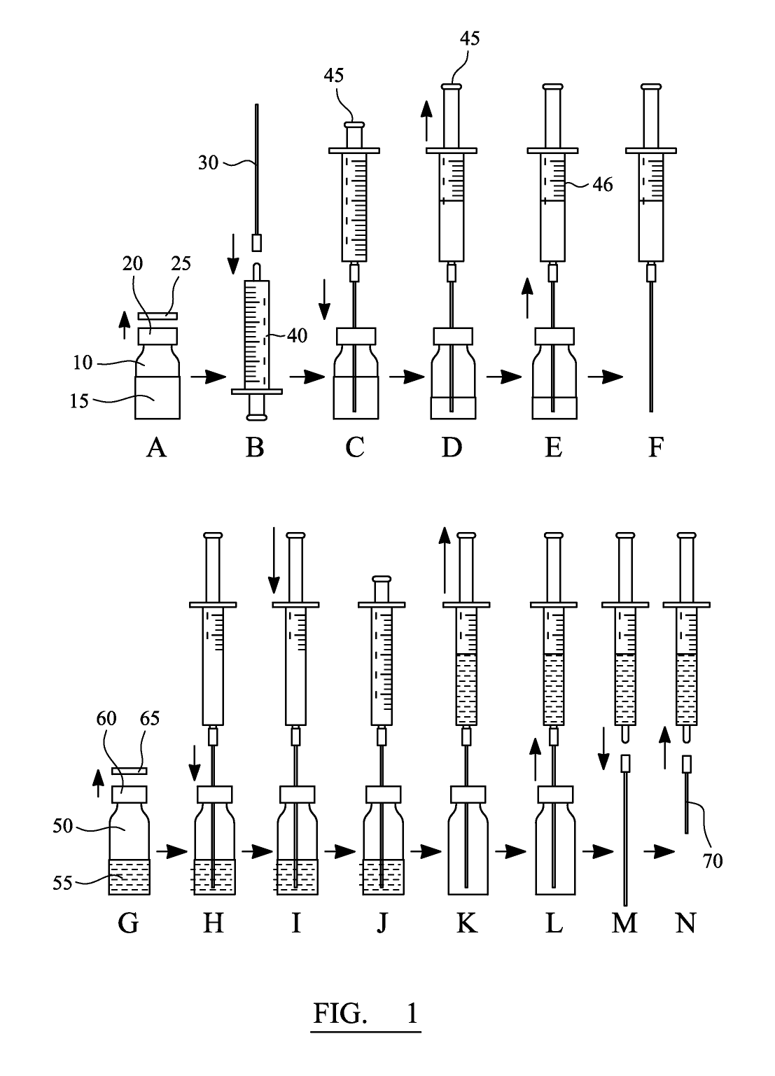

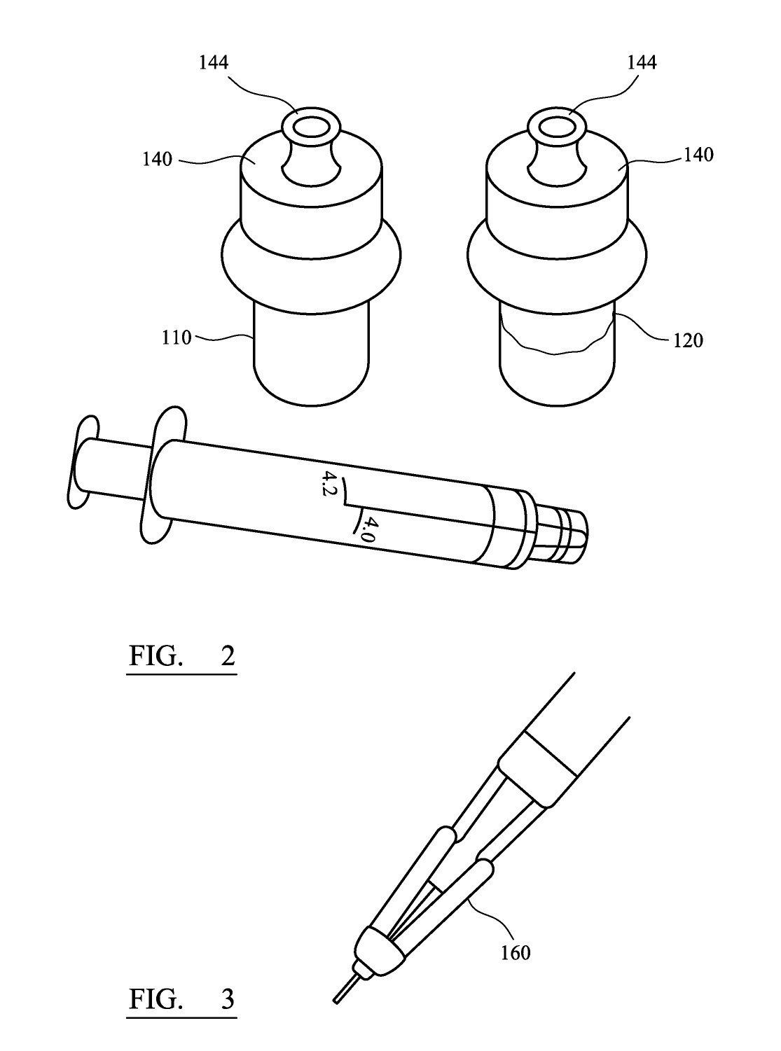

FIGS. 2 to 6 illustrate a first embodiment of a kit for the preparation and administration of a Degarelix solution. The kit may include first and second chambers or vials 110, 120, first and second vial adaptors 140, a syringe 130, a mixing apparatus 150, and a hypodermic needle 160. In an exemplary embodiment, the kit may be configured for the preparation and administration of a 40 mg/ml Degarelix solution comprising a vial containing 6 ml of WFI, a vial containing 88 mg of lyophilised Degarelix to which 4.2 ml of WFI may be added, a syringe, two Medimop VF vial adapters, a V-3 vortex mixer adapted to receive the vials, and a West Pharmaceuticals NOVAguard® safety needle. It is contemplated that any suitably compatible components could be used to make up the kit, and different amounts of WFI and Degarelix may be used. For example, to make up a 40 mg/ml dose the kit may comprise a vial containing 120 mg of Degarelix to which 3 ml of WFI may be added. Likewise, to make up a 60 mg/ml concentration, the kit may comprise a vial containing 180 mg of Degarelix to which 3 ml of WFI may be added. A 20 mg/ml solution is formed by adding 4.2 ml of WFI to a vial containing 88 mgt of Degarelix.

In the first embodiment (see FIG. 2), the syringe 130 may be marked with two lines configured to enable a user to draw, in a first instance, a given amount of WFI into the syringe and, in a second instance, draw a dose of solution into the syringe. In an exemplary embodiment, one line may denote 4.2 ml, which may enable the user to draw 4.2 ml of WFI into the syringe for reconstituting the lyophilized Degarelix. The other line may denote 4 ml, which may enable a user to draw a dose of 4 ml of solution into the syringe for administration to the patient. It is contemplated that different markings could be incorporated on the syringe, for example a syringe for use in making up a 40 mg/ml concentration a syringe may be marked with 3 ml.

It should be noted that forming a solution of 88 mg of lyophilized Degarelix and 4.2 ml of water provides the same strength solution as would be formed using 80 mg Degarelix and 4 ml water. It is contemplated that the excess solution formed by using 4.2 ml of WFI may improve the accuracy of providing a dose of 4 ml of the solution to a patient.

Each vial may be closed by a penetratable rubber stopper. The vial adapters 140 may be designed to be attached to the vials and penetrate the rubber stopper, after which a syringe may be attached to a coupling 144 on the vial adaptors 140. The syringe may be separably couplable to each vial adapter 140 such that there can be communication between each vial and the chamber of the syringe.

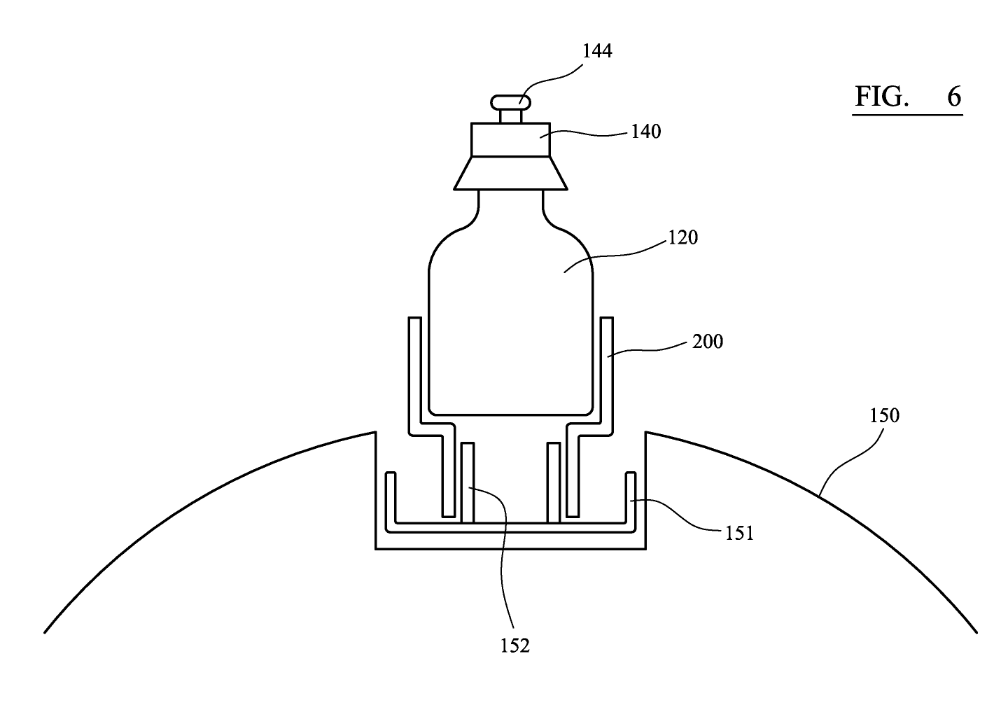

As illustrated in FIG. 6, the vortex mixer may be adapted for receiving a vial, e.g., vials 110 or 120, by the addition of a vial guide sleeve 200 configured to allow the vial to be seated on the mixer. The mixer 150 may include a plate 151 that rotates and a cylinder 152 fixed to the plate.

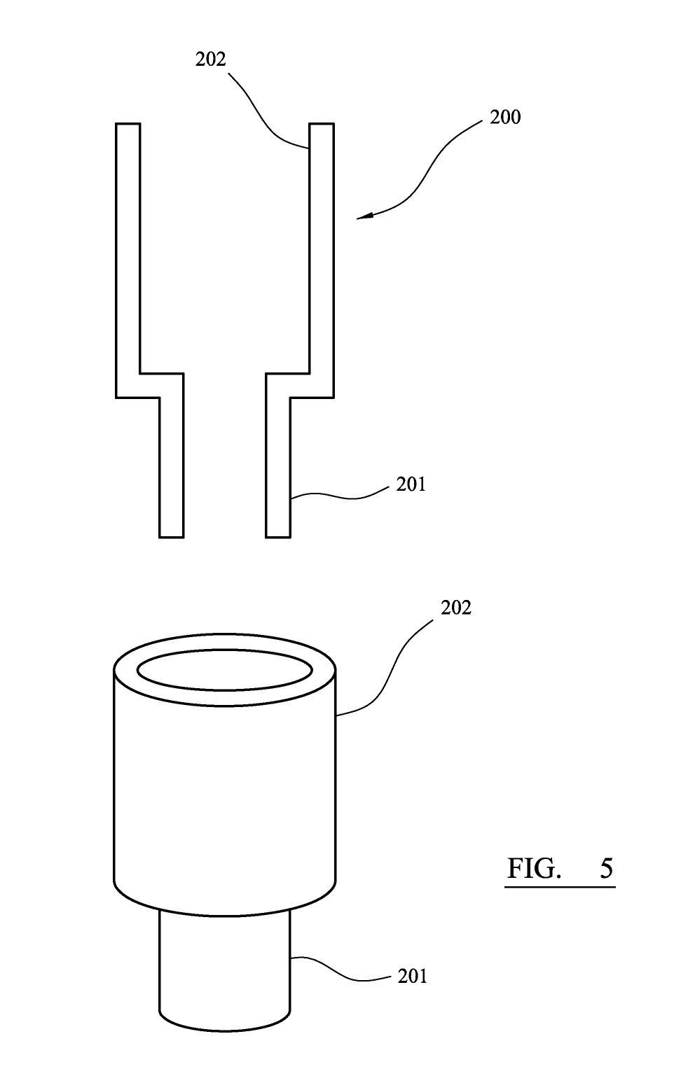

As illustrated in FIGS. 5 and 6, guide sleeve 200 may be a single element having a first end 201 in the form of a cylinder sized to engage with the cylinder 152 of the vortex mixer 150. The second end 202 of the guide sleeve may be in the form of a cylinder sized to receive the vials 110 or 120. When positioned on the vortex mixer, the guide sleeve may allow a vial to be seated on the mixer such that the contents of the vial can be mixed.

As illustrated in FIG. 4, the mixer may also include an intensity setting dial 210 and an indication of an optimum intensity 220 for mixing the Degarelix product.

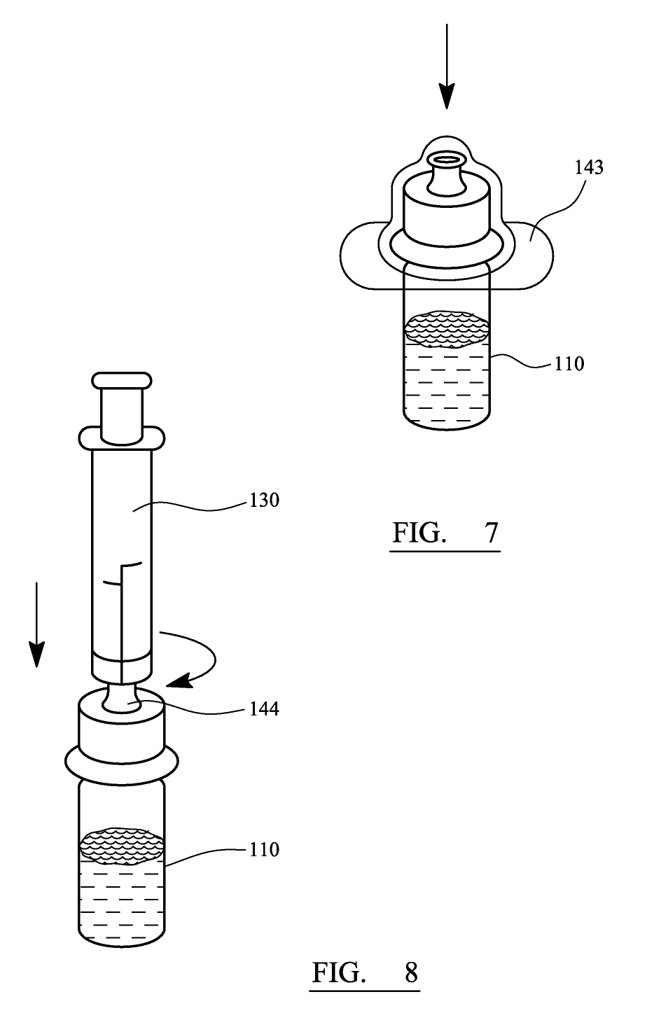

An exemplary method of use of the first embodiment will now be described with reference to FIGS. 7 to 11.

As a first step, the user attaches the vial adapters 140 onto the WFI vial 110 and the lyophilised Degarelix vial 120. Each vial adapter 140 may be seated on the vial by any conventional method. For example, each vial adaptor may be seated by pushing down until a spike (conventionally know and thus not illustrated) in the adapter to penetrate the rubber stopper on the vial and the adapter snaps in place. Additionally the vial adaptors 140 may include covers 143 (conventionally known and thus not further described) protecting the vial adaptor couplings 144. The covers 143, if present, may be removed after attachment of the adaptor to the vial. It is contemplated that the covers 143 may improve sterility of the vial adaptors 140 and in particular the vial adaptor couplings 144.

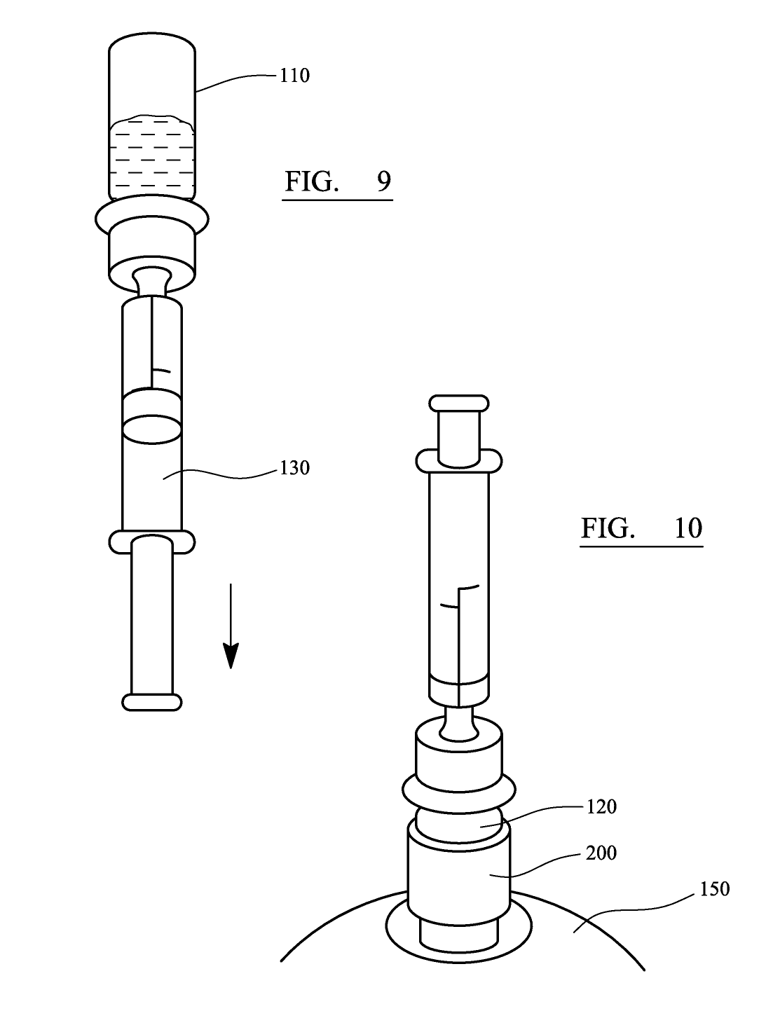

The syringe 130 may be removed from packaging, if present, and attached to the WFI vial 110 via a coupling 144. As is know in the art, attachment may occur by pressing the syringe into the coupling 144 and twisting to engage threads thereon. The WFI vial 110 may be turned upside down and a user may draw water into the syringe (see FIG. 9). In an exemplary embodiment, a user may draw 4.2 ml of water into the syringe.

The syringe, now containing water for injection, may be removed from the WFI vial 110 and connected to the lyophilised Degarelix vial 120. The 4.2 ml of water for injection may then be injected into the lyophilised Degarelix vial 120.

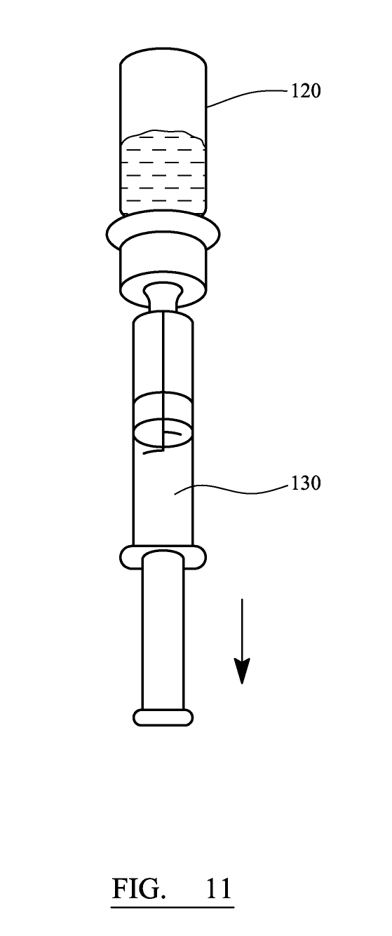

The lyophilised Degarelix vial may be transferred to the vortex mixer 150 and seated within the adapter 200 on the vortex mixer (see FIG. 10). The syringe 130 and vial adapter 140 may remain coupled to the vial during the mixing process. It is contemplated that this may prevent contamination of the contents of the vial during mixing. The vortex mixer may be switched on and the intensity increased to the marked level for mixing the Degarelix. In an exemplary embodiment, the vortex mixer may mix the contents of the lyophilised Degarelix vial 120 for a period of approximately 1.25 minutes. It is contemplated that after the lyophilised Degarelix vial is mixed for this amount of time, there is a high probability that the reconstitution will be complete. It is noted that the time period prescribed for mixing may vary depending on various factors, such as the strength of solution being made and the ratio of lyophilised Degarelix to WFI that needs to be reconstituted. It is contemplated that exemplary prescribed times for mixing are within a range from 1 to 3 minutes.

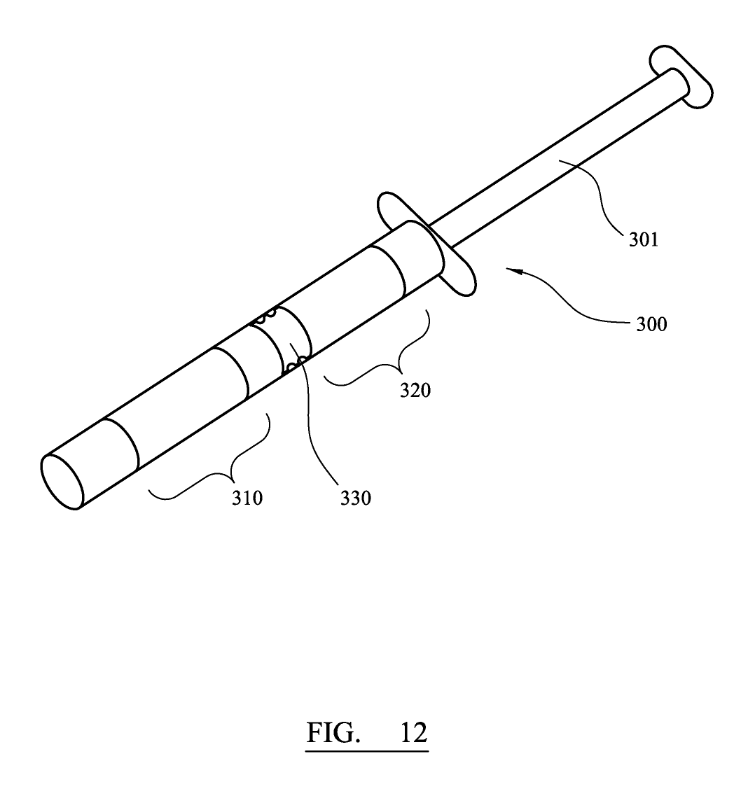

After the prescribed time period has passed, the vortex mixer may be switched off and the vial/syringe assembly may be removed from the mixer. The care-provider may check the Degarelix solution for clarity to determine whether the reconstitution is suitably complete. If so, the lyophilised Degarelix vial 120 may then be turned upside down and a dosage amount of the Degarelix solution may be drawn into the syringe (see FIG. 11). In an exemplary embodiment, a dosage amount of about 4 ml of the Degarelix solution may be drawn into the syringe.

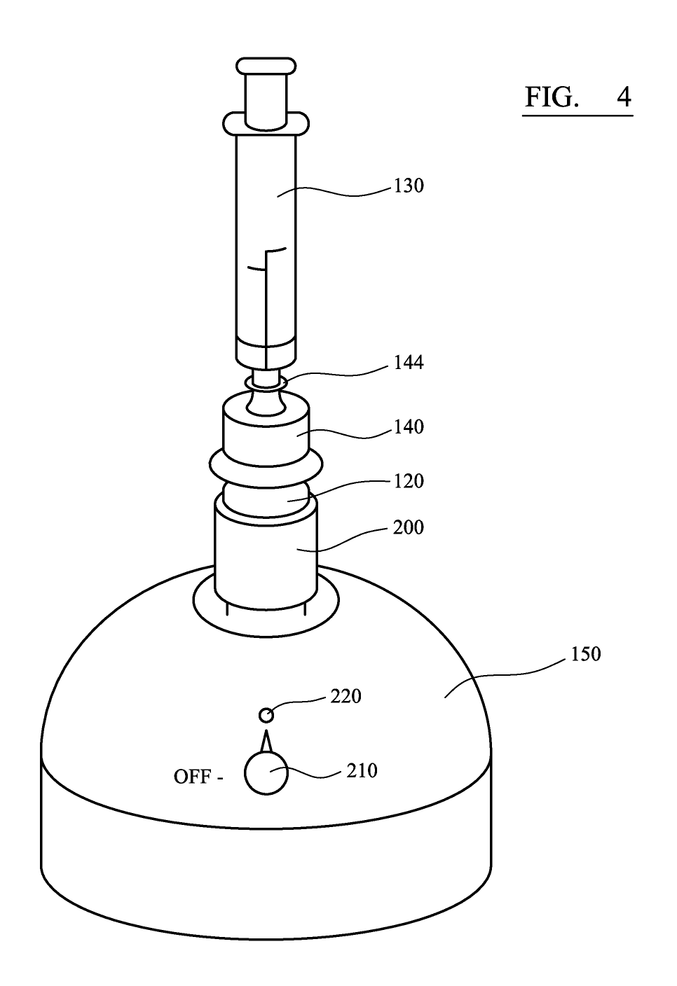

The syringe may be detached from the vial adapter 140 and attached to the safety needle 160 (see FIG. 3). Air bubbles may be removed and a subcutaneous injection may be performed, inserting the needle deeply into a patient at an angle not less than approximately 45 degrees.

FIG. 12 illustrates a second embodiment of a kit for the preparation and administration of a Degarelix solution. The kit of the second embodiment may include a dual-chambered syringe 300 preloaded with WFI and lyophilised Degarelix product. The syringe may include two chambers, a first chamber 310 containing the lyophilised Degarelix and a second chamber 320 containing the WFI. The kit may also comprise a vortex mixer adapted to receive the dual chambered syringe and a safety needle device for performing subcutaneous injection into the patient similar to the vortex mixer 150 and the safety needle 160 described above with respect to the first embodiment of the kit. As such, these components of the second embodiment are not further described in detail.

An exemplary method of use of the second embodiment of the kit will now be described. The syringe 300 may be activated by depressing its plunger 301 to force the WFI in the second chamber into the first chamber containing the lyophilised Degarelix product. The water may pass through a port 330 between the two chambers in a known manner. For example, the water may be transferred from the second chamber to the first chamber under pressure, that is, as a pressurized stream. The syringe 300 may then be placed within the adapter on the vortex mixer and the mixer may be switched on to the appropriate intensity to mix the lyphilised Degarelix solution.

In an exemplary embodiment, the solution may be mixed for approximately 1.25 minutes after which a care-provider may check the Degarelix solution for clarity to determine whether the reconstitution is suitably complete. Similar to the first embodiment, a safety needle may be coupled to the dual chambered syringe 300, air bubbles may be removed, and the dose may then be delivered directly to the patient.

It is contemplated that the dual-chambered syringe 300 may enable the WFI to be directly transferred from the second chamber to the first chamber. It is also contemplated that that the solution may be administered to a patient directly from the first chamber. These features of the second embodiment may decrease the time taken to prepare and administer the drug and may reduce the potential for contamination.

The following description of Degarelix doses are applicable to the kit embodiments and/or their respective method of use.

A dual chambered syringe may be used for preparing different concentrations of Degarelix solution. For example, the first chamber may contain 80 mg of lyophilised Degarelix and the second chamber my contain 4 ml of WFI, the resulting Degarelix solution having a concentration of 20 mg/ml.

Alternatively, the first chamber may contain 120 mg of lyophilised Degarelix and the second chamber my contain 3 ml of WFI, the resulting Degarelix solution having a concentration of 40 mg/ml.

As a further example, the first chamber may contain 180 mg of lyophilised Degarelix and the second chamber my contain 3 ml of WFI, the resulting Degarelix solution having a concentration of 60 mg/ml.

Kits according to any embodiment described above may be used for preparing Degarelix solutions for the treatment of prostate cancer. For those treatments, it is contemplated that a Degarelix solution is injected into a patient as soon as possible after reconstitution.

A solution concentration of 40 mg/ml may be used to deliver a starter dose for a patient beginning a course of treatment for advanced prostate cancer (for example, two injections each containing 3 ml of solution at concentration of 40 mg/ml may be administered as a starting dose). In this case a total 240 mg dose of drug would be administered. After this initial starter dose maintenance doses may be delivered at a lower concentration (for example as single injections of 4 ml of solution at concentration of 20 mg/ml, giving a total maintenance dose of 80 mg). It is noted that different dosing regimes may apply in different situations. For example, the starter dose may be higher, lower or the same as the maintenance dose.

In order to facilitate preparation of Degarelix solution at different concentrations for administration of different doses, a kit accordingly may contain vials of WFI, vials of Degarelix drug product, and syringes marked suitably for preparation of different doses, for example both a starter dose and a maintenance dose of Degarelix.

In order to facilitate preparation of Degarelix solution at different concentrations for administration of different doses, a kit accordingly may comprise separate dual-chambered syringes containing different amounts of lyophilised Degarelix and/or different volumes of WFI to enable reconstitution of solutions having different concentrations.

Additionally, the kit embodiments may include an exemplary dual chamber syringe having 30 mg of Degarelix in the first chamber and 3 ml's of water in the second chamber. It is contemplated that such an exemplary dual chamber syringe may enable the preparation of 30 mg of a Degarelix solution at a concentration of 40 mg/ml by transferring 0.75 ml of water from the second chamber to the first chamber. That embodiment may further include another exemplary dual chamber syringe having 20 mg of Degarelix in the first chamber and 1 ml of water in the second chamber. It is contemplated that such an exemplary dual chamber syringe may enable the preparation of 20 mg of Degarelix solution at a concentration of 40 mg/ml by transferring 0.5 ml of water from the second chamber into the first chamber.

It will be apparent to those skilled in the art that various modifications and variations can be made to the disclosed apparatus and methods. Other embodiments will be apparent to those skilled in the art from consideration of the specification and practice of the disclosed system. It is intended that the specification and examples be considered as exemplary only, with a true scope being indicated by following claims and their equivalents.

What is claimed is:

1. A kit for the preparation of a Degarelix solution for administration to a patient, comprising:
a first chamber containing a mass of lyophilised Degarelix;
a second chamber containing a volume of water;
a syringe configured to transfer at least a portion of the volume of water from the second chamber to the first chamber;
a mixing apparatus comprising a mixer and an adaptor configured for receiving at least the first chamber; and
at least one of a hypodermic needle, a safety needle apparatus or a needleless apparatus, each of which is configured for subcutaneous delivery of a solution to a patient.

2. The kit according to claim 1, further comprising:
a first vial defining the first chamber;
a second vial defining the second chamber;
first and second vial adaptors configured to provide needleless communication between a respective one of the first and second vials and the syringe;
wherein the syringe is configured to be selectably connected to each of the first and second vials via a respective one of the first and second vial adaptors.

3. The kit according to claim 2, wherein the syringe is further configured to withdraw an amount of water from the second chamber, insert at least a portion of the amount of water to the first chamber, and withdraw a portion of a solution of Degarelix and water from the first chamber.

4. The kit according to claim 1, wherein the adaptor includes a guide sleeve configured to support the syringe when the first chamber is received in the adaptor.

5. The kit according to claim 1, wherein the syringe is further configured to inject the at least a portion of the volume of water from the second chamber to the first chamber as a pressurized stream.

6. The kit according to claim 1, wherein the lyophilized Degarelix of the first chamber ranges from 10 mg to 300 mg.

7. The kit according to claim 1, wherein the syringe is the second chamber containing the volume of water.

8. A kit for the preparation of a Degarelix solution for administration to a patient, comprising:
a multi-chambered syringe having a first chamber containing a mass of lyophilised Degarelix, a second chamber containing a volume of water, for injection and a port disposed between the first and second chambers configured to selectively transfer at least a portion of the volume of water from the second chamber to the first chamber;
a mixing apparatus comprising a mixer and an adaptor configured for receiving the multi-chamber syringe; and
at least one of a hypodermic needle, a safety needle apparatus or a needleless apparatus, each of which is configured for subcutaneous delivery of a solution to a patient.

9. The kit according to claim 8, wherein lyophilized Degarelix of the first chamber ranges from 10 mg to 300 mg.

10. A kit for the preparation of a Degarelix solution for administration to a patient, comprising:
at least two first chambers, one of the at least two chambers containing a first predetermined mass of lyophilized Degarelix for preparing a Degarelix solution with a first concentration and another of the at least two first chambers containing a second predetermined mass of lyophilized Degarelix different from the first predetermined mass of Degarelix for preparing a Degarelix solution with a second concentration.

11. A method of preparing a Degarelix solution for administration to a patient comprising:
transferring a volume of water from a first chamber into a second chamber containing a mass of lyophilised Degarelix;
connecting the second chamber to a mixing apparatus and mixing the transferred volume of water and the mass of lyophilised Degarelix for a period of time to reconstitute the Degarelix into a solution;
disconnecting the second chamber from the mixing apparatus after the period of time.

12. The method of claim 11, further comprising transferring the volume of water from the first chamber to the second chamber as a pressurized stream.

13. The method of claim 11, further comprising transferring the volume of water from the first chamber to the second chamber so as to create turbulent mixing with the lyphiloised Degarelix.

14. The method of claim 11, wherein the period of time is less than approximately 5 minutes.

15. The method of claim 11, wherein the period of time is between approximately 0.5 minutes and approximately 3 minutes.

16. The method of claim 11, wherein the period of time is between approximately 0.8 minutes and approximately 1.5 minutes.

17. The method of claim 11, wherein the period of time is approximately 1.25 minutes.

18. The method of claim 11, further comprising connecting the second chamber to the mixing apparatus via an adaptor, wherein the adapter includes a first portion configured to fixedly connect to the mixing apparatus and a second portion configured to receive the first chamber.

19. The method of claim 11, wherein the mixing apparatus is a vortex mixer.

20. The method of claim 11, further including transferring a volume of the Degarelix solution from the second chamber into a chamber of a syringe.

21. The method of claim 11, wherein:
the first chamber and the second chamber are each respective chambers of a multi-chambered syringe; and
connecting the second chamber to the mixing apparatus includes connecting the multi-chambered syringe to the mixing apparatus.

22. The method of claim 21, wherein transferring the volume of water includes injecting the volume of water from the first chamber into the second chamber via a port disposed within the multi-chambered syringe.

23. The method of claim 11, wherein:
the first chamber is defined by a first vial;
the second chamber is defined by second vial; and
connecting the second chamber to the mixing apparatus includes connecting the second chamber to the mixing apparatus.

24. The method of claim 23, wherein transferring the volume of water comprises:
connecting a single-chambered syringe to the first vial via a first vial adaptor;
withdrawing the volume of water from the first vial;
disconnecting the single-chambered syringe from the first vial;
connecting the single-chambered syringe to the second vial via a second vial adaptor; and
injecting the volume of water into the second vial.
